(19) United States
(12) Patent Application Publication
(10) Pub. No.: US 2025/0186535 A1
(43) Pub. Date: Jun. 12, 2025

(54) OPHTALMIC COMPOSITIONS AND METHODS OF USING SAME

(71) Applicant: Tarsier Pharma Ltd., Zichron Yaacov (IL)

(72) Inventors: Zohar MILMAN-LEVINSON, Kibbutz Tel Yosef (IL); Daphne HAIM-LANGFORD, Zichron Yaacov (IL); Olivier Hendrik DEFERT, Le Havre (FR)

(21) Appl. No.: 18/693,656

(22) PCT Filed: Sep. 22, 2022
(86) PCT No.: PCT/IL2022/051018

§ 371 Date: Mar. 20, 2024

Related U.S. Application Data
(60) Provisional application No. 63/247,404, filed on Sep. 23, 2021.

(51) Int. Cl. A61K 38/07; A61K 9/00; A61K 47/18; A61K 47/36; A61P 27/02

(52) U.S. Cl. CPC A61K 38/07; A61K 9/0048; A61K 47/186; A61K 47/36; A61P 27/02

(57) ABSTRACT

Provided herein is an ophthalmic composition, comprising an aqueous solution, comprising (i) a pharmaceutically effective amount of a phosphorylcholine-tuftsin conjugate, (ii) between 0.1 and 5% weight per weight (w/w) of a viscosity enhancer, and (iii) between 0.1 and 5% w/w of a mucoadhesive polymer. Provided herein is a synergistic preservative composition comprising a phosphorylcholine-tuftsin conjugate and BAK. Furthermore, a pharmaceutical composition (e.g. ophthalmic composition) comprising an effective amount of the preservative composition is also povided. A method of using the ophthalmic composition such as for treating or preventing ocular inflammation in a subject in need thereof is also provided. Furthermore, method of using the ophthalmic composition such as for enhancing ocular bioavailability of a phosphorylcholine-tuftsin conjugate within a subject is also provided.

### CROSS REFERENCE TO RELATED APPLICATIONS

This application claims the benefit of priority of U.S. Provisional Patent Application No. 63/247,404, filed on Sep. 23, 2021, the content of which is incorporated herein by reference in its entirety.

### FIELD OF INVENTION

This invention is directed to ophthalmic compositions and uses thereof, such as for encapsulation and ocular delivery of therapeutically active agents.

### BACKGROUND OF THE INVENTION

Ocular inflammation, an inflammation of any part of the eye, is one of the most common ocular diseases. Ocular inflammation refers to a wide range of inflammatory disease of the eye, one of them is uveitis. These diseases are prevalent in all age groups and may be associated with systemic diseases such as Crohn's disease, Behcet disease, Juvenile idiopathic arthritis and others. The inflammation can also be associated with other common eye symptoms such as dry eye and dry macular degeneration. Several drugs have the known side effect of causing uveitis and/or dry eye. The most common treatment for ocular inflammation, is steroids and specifically corticosteroids. However, these treatments have several known and sometimes severe side effects.

Tuftsin-PhosphorylCholine (TPC) is bi-specific small molecule with immunomodulatory activities. Tuftsin (Thr-Lys-Pro-Arg, SEQ ID NO:1) is a natural immunomodulating peptide produced by enzymatic cleavage of the Fc-domain of the heavy chain of IgG in the spleen. Phosphorylcholine is a small zwitterionic molecule secreted by helminths which permits helminths to survive in the host inducing a situation of immune tolerance as well as on the surface of some bacteria and apoptotic cells.

Methods of treating ocular inflammation are greatly needed. Additionally, formulations of Tuftsin-Phosphoryl Choline for direct administration to the eye are greatly needed.

### SUMMARY OF THE INVENTION

In one aspect of the invention, there is an ophthalmic composition comprising an aqueous solution, comprising (i) a pharmaceutically effective amount of a phosphorylcholine-tuftsin conjugate, (ii) between 0.1 and 5% weight per weight (w/w) of a viscosity enhancer, and (iii) between 0.1 and 5% w/w of a mucoadhesive polymer.

In one embodiment, a w/w ratio between the mucoadhesive polymer and the viscosity enhancer is between 1:1 and 10:1.

In one embodiment, the pharmaceutically effective amount is between 0.01 and 10% w/w of the phosphorylcholine-tuftsin conjugate.

In one embodiment, the phosphorylcholine-tuftsin conjugate comprises at least one phosphorylcholine moiety or a derivative thereof and tuftsin or a derivative thereof covalently linked via a spacer.

In one embodiment, the spacer comprises at least two amino acids.

In one embodiment, the phosphorylcholine moiety or a derivative thereof is covalently linked to the spacer via a diazo group.

In one embodiment, the phosphorylcholine-tuftsin conjugate is represented by Formula 1 (SEQ ID NO: 2):

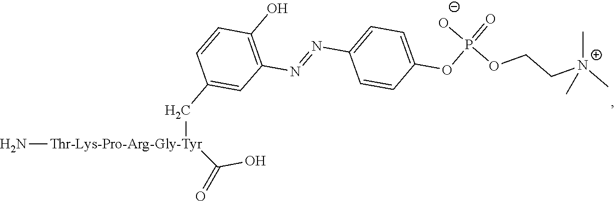

including any salt thereof.

In one embodiment, the mucoadhesive polymer comprises an ionic mucoadhesive polymer.

In one embodiment, the ionic mucoadhesive polymer is selected form the group consisting of: alginate, chitosan, polyacrylate, hyaluronic acid, carboxymethylcellulose, pectin, and gelatin including any salt or any combination thereof.

In one embodiment, the viscosity enhancer comprises hydroxy methyl cellulose, hydroxy ethyl cellulose, cellulose, hydroxypropyl methyl cellulose, polyvinylpyrrolidone, polyethyleneglycol, polypropyleneglycol, poly(2-hydroxyethyl methacrylate), and polyvinylalcohol or any combination thereof.

In one embodiment, the aqueous solution comprises a pharmaceutically acceptable salt.

In one embodiment, the ophthalmic composition further comprises up to 5% w/w of any one of a preservative, a tonicity regulator.

In one embodiment, the ophthalmic composition is in a form of an eye drop formulation.

In another aspect, there is a method for treating or preventing ocular inflammation in a subject in need thereof, the method comprising administering to an eye of the subject an effective amount of the ophthalmic composition of the invention.

In one embodiment, the ocular inflammation is uveitis, or associated with any one of dry eye, dry macular degeneration, and post operation inflammation.

In another aspect, there is a method for enhancing ocular bioavailability of a phosphorylcholine-tuftsin conjugate in a subject, comprising administering to an eye of the subject the ophthalmic composition of the invention.

In one embodiment, enhancing ocular bioavailability is by at least 10% compared to a control.

In one embodiment, enhancing ocular bioavailability comprises increasing concentration of the phosphorylcholine-tuftsin conjugate in an aqueous humor of the eye.

In one embodiment, the subject is selected from a human subject and an animal subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DETAILED DESCRIPTION OF THE INVENTION

The present invention, in some embodiments thereof, relates to an ophthalmic composition comprising phosphorylcholine-tuftsin conjugate (PC), and to a method for treating medical conditions by administering the composition to a subject in need thereof.

The present invention, in further embodiments thereof, relates to an ophthalmic composition for ocular administration and to use thereof in the treatment of medical conditions related to an ocular inflammation in a subject in need thereof.

The present invention, in some embodiments thereof, relates to a preservative composition comprising the PC and a quaternary ammonium cation and/or any salt thereof, wherein a w/w ratio between the PC and the quaternary ammonium cation within the preservative is at least about 1000:1, and wherein the preservative is characterized by an antimicrobial activity within an aqueous composition at an effective concentration corresponding to a concentration of the quaternary ammonium cation being reduced by at least 10%, relative to an effective concentration of the quaternary ammonium cation within the same aqueous composition devoid of PC.

The present invention, in some embodiments thereof, relates to a methodology for encapsulating active agents in an amphiphilic polymer comprising various hydrophobic and hydrophilic domains within the same particle. The polymer may have nano-sized or submicron-sized structure. Using this methodology, polymeric structures encapsulating drugs have been prepared and characterized. Exemplary compositions significantly enhanced the in-vivo bioavailability of the drug.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Composition

In one aspect of the invention, there is a pharmaceutical composition comprising (i) an aqueous solution comprising a phosphorylcholine-tuftsin conjugate, (ii) between 0.1 and 5% weight per weight (w/w) of a viscosity enhancer, and (iii) between 0.1 and 5% w/w of a mucoadhesive polymer. In some embodiments, the pharmaceutical composition is a liquid composition. In some embodiments, the pharmaceutical composition is formulated for ocular administration. In some embodiments, the pharmaceutical composition is an ophthalmic composition. In some embodiments, the terms “ophthalmic composition” and “pharmaceutical composition” are used herein interchangeably.

In some embodiments, the ophthalmic or the pharmaceutical composition is a liquid at a temperature between −5 and 95° C. In some embodiments, the ophthalmic composition is a liquid under physiological conditions (e.g. a temperature between 2° and 40° C., a pH between 2 and 8, etc.) In some embodiments, the ophthalmic composition is an aqueous composition. In some embodiments, the ophthalmic composition is a solution (e.g. an aqueous solution). In some embodiments, the components of the ophthalmic composition are substantially soluble within the solution. In some embodiments, the ophthalmic composition is substantially devoid of any particulate matter. In some embodiments, the ophthalmic composition is substantially devoid of particles having a particle size greater than 200 nm, 300 nm, 400 nm, 500 nm, 600 nm, 700 nm, 800 nm, 900 nm, 1 μm, including any range or value therebetween. In some embodiments, the ophthalmic composition is a clear solution. In some embodiments, the ophthalmic composition is an opaque solution.

In some embodiments, the ophthalmic composition is characterized by transmittance of more than 80%, more than 85%, more than 90%, more than 95%, more than 97%, more than 99%, wherein the transmittance is measured at wavelengths ranging from 200 nmn to 1000 nm. One skilled in the art will appreciate that transmittance is an indication for optical clarity ofthe ophthalmic composition (e.g. in a form of a solution).

In some embodiments, the ophthalmic composition is a colored solution. In some embodiments, the colored solution is characterized by a slight yellow to red color.

In some embodiments, the ophthalmic composition comprises a solvent. In some embodiments, the solvent is an aqueous solvent. In some embodiments, the ophthalmic composition comprises between 50 and 99%, between 50 and 60%, between 60 and 70%, between 70 and 80%, between 80 and 85%, between 85 and 90%, between 90 and 95%, between 90 and 92%, between 92 and 95%, between 95 and 97%, between 97 and 99%, w/w of the solvent including any range or value therebetween.

In some embodiments, the ophthalmic composition is an aqueous solution. In some embodiments, the aqueous solution is a buffered solution. In some embodiments, the aqueous solution comprises a pharmaceutically acceptable salt. In some embodiments, the buffered solution comprises a phosphate buffer. In some embodiments, the aqueous solution comprises water for injection, which is well-known in the art.

Among suitable buffer components or buffering agents that may be employed in the aqueous solution, are those conventionally used in ophthalmic compositions. In some embodiments, pharmaceutically acceptable salt within a buffered solution include alkali metal, alkaline earth metal and/or ammonium salts, as well as citrate, phosphate, borate, lactate and the like salts and mixtures thereof. Conventional organic buffers, such as Goode's buffer and the like, may also be employed.

In some embodiments, the ophthalmic composition comprises a therapeutically effective amount of the phosphorylcholine-tuftsin conjugate. In some embodiments, the ophthalmic composition comprises the phosphorylcholine-tuftsin conjugate, a pharmaceutically acceptable salt thereof or both.

The term “therapeutically effective amount” refers to the amount of the conjugate effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition. In some embodiments, the term “therapeutically effective amount” and the term “pharmaceutically effective amount” are used herein interchangeably.

In some embodiments, a weight per weight (w/w) concentration of the phosphorylcholine-tuftsin conjugate within the ophthalmic composition is between 0.01 and 10%, between 0.01 and 0.03%, between 0.03 and 0.5%, between 0.5 and 0.7%, between 0.7 and 0.8%, between 0.8 and 1%, between 1 and 1.5%, between 1.5 and 2%, between 2 and 2.5%, between 2.5 and 3%, between 3 and 4%, between 4 and 5%, between 5 and 7%, between 7 and 10%, including any range or value therebetween. In some embodiments, the pharmaceutically effective amount of the phosphorylcholine-tuftsin conjugate within the ophthalmic composition is between 0.01 and 10%, between 0.1 and 1%, between 1 and 5% by weight.

In some embodiments, the ophthalmic composition comprises 0.1%, 0.5%, 1%, 1.5%, 2%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%1, 5%, 20%, 30%, or about 40% of phosphorylcholine-tuftsin conjugate, including any value and range therebetween, by total dry weight of the ophthalmic composition.

In some embodiments, the ophthalmic composition comprises the phosphorylcholine-tuftsin conjugate as the only pharmaceutically active ingredient. In some embodiments, the ophthalmic composition is substantially devoid of any additional pharmaceutically active ingredient. In some embodiments, the ophthalmic composition is substantially devoid of any additional anti-inflammatory agent.

In some embodiments, the phosphorylcholine-tuftsin conjugate comprises at least one phosphorylcholine moiety or a derivative thereof and tuftsin or a derivative thereof covalently linked via a spacer. In some embodiments, the spacer comprises at least two amino acids. In some embodiments, the phosphorylcholine moiety or a derivative thereof is covalently linked to the spacer via a diazo group.

The term “phosphorylcholine (PC) conjugate” as used herein, refers to a phosphorylcholine moiety or a derivative thereof linked to tuftsin (T), optionally via a spacer.

As used herein, the term “tuftsin” refers to a tetra-peptide (threonine-lysine-proline-arginine, TKPR; SEQ ID NO: 1). Tuftsin may be synthesized chemically. Tuftsin is known for its phagocytosis-stimulating activity and augmentation of antigen presenting capacity of macrophages in-vitro and in-vivo. According to some embodiments, tuftsin may be considered as an immunomodulatory molecule.

The term “derivative of phosphorylcholine” as used herein, refers to any compound that is based off phosphorylcholine. The term “derivative of tuftsin” as used herein, refers to any polypeptide that is based off of TKPR. In some embodiments, the derivative retains the immunomodulatory effects of phosphorylcholine and/or tuftsin. In some embodiments, the derivative is a derivative comprising phosphorylcholine. In some embodiments, the derivative is a derivative comprising TKPR. A derivative is not merely a fragment of the polypeptide, nor does it have amino acids replaced or removed (an analog), rather it may have additional modification made to the polypeptide, such as a post-translational modification.

In some embodiments, the derivative of phosphorylcholine is selected from: 4-amino-phenyl-phosphocholine, 4-diazonio-phenyl-phosphorylcholine, 4-nitro-phenyl-phosphocholine and 12-(3-iodophenyl)dodecyl-phosphocholine among others. Each possibility is a separate embodiment of the invention.

The terms “tuftsin derivative”, “TD” and “tuftsin-derived carrier moiety” are interchangeable and refer to tuftsin (TKPR, SEQ ID NO: 1) attached to at least two additional amino acids which are independently selected. Non-natural amino acids, preferably non-charged and non-polar non-natural amino acids such as 0-alanine-6-aminohexanoic acid and 5-aminopentanoic acid, may also be comprised in the tuftsin derivative. In some embodiments, the tuftsin derivative is Threonine-Lysine-Proline-Arginine-Glycine-Tyrosine (TKPRGY, SEQ ID NO: 2).

The term “moiety” as used herein refers to a part of a molecule, which lacks one or more atom(s) compared to the corresponding molecule. The term “moiety”, as used herein, further relates to a part of a molecule that may include either whole functional groups or parts of functional groups as substructures. The term “moiety” further means part of a molecule that exhibits a particular set of chemical and/or pharmacologic characteristics which are similar to the corresponding molecule.

The terms “linked” or “attached” as used herein refer to a bond between at least two molecules or moieties such that they are a single molecule. In some embodiments, the bond is a chemical bond. In some embodiments, the bond is a covalent bond.

According to the principles of the present invention, the natural and non-natural amino-acids comprised in the tuftsin derivative are adjacent and attached to one another, while the at least one phosphorylcholine derivative is attached to the at least one tuftsin derivative either directly or indirectly via a spacer. In some embodiments, the at least one phosphorylcholine or derivative thereof is linked to the N-terminus of at least one tuftsin or derivative thereof. In some embodiments, the at least one phosphorylcholine or derivative thereof is linked to the C-terminus of at least one tuftsin or derivative thereof.

The term “spacer”, as used herein, refers to a connecting or otherwise bridging element between the tuftsin derivative and the phosphoryl choline derivative, typically linked by chemical methods or biological means thereto. Non-limiting examples of spacers include: amino acids, peptides, polypeptides, proteins, hydrocarbons and polymers among others. Each possibility is a separate embodiment of the invention. In some embodiments, the spacer is at least 2 amino acids. In some embodiments, the spacer is Glycine-Tyrosine. In some embodiments, the spacer is attached to the C-terminus of TKPR. In some embodiments, the spacer is attached to the N-terminus of TKPR.

In some embodiments, the phosphorylcholine-tuftsin conjugate described above comprises one phosphorylcholine derivative attached to one tuftsin derivative. In certain embodiments, the phosphorylcholine-tuftsin conjugate described above comprises a plurality of phosphorylcholine derivatives attached to a plurality of tuftsin derivatives. In certain embodiments, the phosphorylcholine-tuftsin conjugate described above comprises a plurality of tuftsin derivatives attached to one phosphorylcholine derivative. In certain embodiments, the phosphorylcholine-tuftsin conjugate described above comprises a plurality of phosphorylcholine derivatives attached to one tuftsin derivative.

In some embodiments, the phosphorylcholine-tuftsin conjugate described above comprises at least one phosphorylcholine or derivative thereof and the at least one tuftsin or derivative thereof separated by a spacer.

In some embodiments, the phosphorylcholine-tuftsin conjugate comprising the tuftsin derivative of SEQ ID NO: 2, is represented by Formula 1:

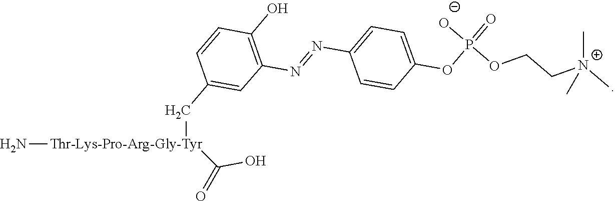

In some embodiments, the phosphorylcholine-tuftsin conjugate is in a form of a salt, comprising a counter ion. In some embodiments, the counter ion is a pharmaceutically acceptable ion. In some embodiments, the counter ion is any of chloride, acetate and trifluoroacetate or any combination thereof.

In some embodiments, the pharmaceutical composition comprises a viscosity enhancer. In some embodiments, the pharmaceutical composition comprises a plurality of viscosity enhancers. In some embodiments, the pharmaceutical composition comprises at least two viscosity enhancers.

In some embodiments, a w/w concentration of a viscosity enhancer within the pharmaceutical composition is between 0.1 and 5%, between 0.1 and 0.2%, between 0.2 and 0.3%, between 0.3 and 0.5%, between 0.4 and 0.6%, between 0.5 and 0.7%, between 0.7 and 0.8%, between 0.8 and 1%, between 1 and 2%, between 2 and 3%, between 3 and 5%, including any range or value therebetween. In some embodiments, a w/w concentration of the viscosity enhancer within the pharmaceutical composition is between 0.4 and 0.6%.

As used herein, a “viscosity enhancer” refers to any substance that increases the viscosity of the solution to be administered to the eye. In some embodiments, the viscosity enhancer increases viscosity of an aqueous solution.

Non-limiting examples of viscosity enhancers include but are not limited to hydroxypropyl methyl cellulose (HPMC), hydroxy methyl cellulose, hydroxy ethyl cellulose, cellulose, polyalcohol polyvinylpyrrolidone, polyethyleneglycol, polypropyleneglycol, polyacrylate, poly(2-hydroxyethyl methacrylate), and polyvinylalcohol or any combination thereof.

Additional viscosity enhancers are well known in the art, such as poloxamer, galactomannan, hydrolyzed galactomannan, glucomannan, hydrolyzed glucomannan, guar gum, xanthan gum, nanocrystalline cellulose, cyclodextrins, poly(cyclodextrins), dextran, dextrin, starch, heparin, beta-glucan, mannan, and levan or any combination thereof.

In some embodiments, the pharmaceutical composition comprises a mucoadhesive polymer. In some embodiments, a w/w concentration of the mucoadhesive polymer within the pharmaceutical composition is between 0.1 and 5%, between 0.1 and 0.2%, between 0.2 and 0.3%, between 0.3 and 0.5%, between 0.4 and 0.6%, between 0.5 and 0.7%, between 0.7 and 0.8%, between 0.8 and 1%, between 1 and 2%, between 2 and 3%, between 3 and 5%, including any range or value therebetween. In some embodiments, a w/w concentration of the mucoadhesive polymer within the pharmaceutical composition is between 1 and 1.5%.

In some embodiments, the mucoadhesive polymer is an ionic mucoadhesive polymer. In some embodiments, the mucoadhesive polymer is a cationic polymer, an anionic polymer or both.

Non-limiting examples of mucoadhesive polymers include, but are not limited to alginic acid, chitosan, hyaluronic acid, carboxymethylcellulose, pectin, fucoidan, dermatan sulfate, chondroitin sulfate, polycarbophil-cysteine, polyoxyethylene glycol ester, heparan sulfate, keratin sulfate, and gelatin, including any salt, any copolymer, or any combination thereof.

In some embodiments, the mucoadhesive polymer is alginic acid (or alginate) and/or a salt thereof.

In some embodiments, a w/w ratio between the mucoadhesive polymer and the viscosity enhancer within the ophthalmic composition is between 1:1 and 10:1, between 1:1 and 2:1, between 2:1 and 4:1, between 2:1 and 3:1, between 3:1 and 5:1, between 3:1 and 4:1, between 5:1 and 7:1, between 7:1 and 10:1, including any range or value therebetween.

In some embodiments, a w/w ratio between the mucoadhesive polymer and the phosphorylcholine-tuftsin conjugate within the ophthalmic composition is between 5:1 and 1:5, between 5:1 and 3:1, between 3:1 and 2:1, between 2:1 and 1:1, between 1:1 and 1:2, between 1:2 and 1:4, between 1:4 and 1:5, including any range or value therebetween.

In some embodiments, the ophthalmic composition comprises up to between 0.1 and 10% w/w of a tonicity regulator.

In some embodiments, the ophthalmic composition comprises an effective amount of a preservative. In some embodiments, the effective amount is a preservative effective amount. In some embodiments, the effective amount is an antimicrobial effective amount. In some embodiments, the effective amount of the preservative within the ophthalmic composition of the invention is reduced relative to the label claim of the preservative (i.e. the effective concentration of the specific preservative as prescribed by the relevant regulatory authority).

In some embodiments, the effective amount of the preservative within the ophthalmic composition of the invention is reduced by at least 10%, at least 50%, at least 70%, at least 2 times, at least 5 times, at least 10 times, at least 50 times, at least 100 times, at least 500 times, or between about 2 and about 20 times, between about 2 and about 15 times, between about 2 and about 10 times, between about 5 and about 20 times, between about 5 and about 15 times, including any range or value therebetween, wherein reduced is relative to the label claim of the preservative. In some embodiments, the ophthalmic composition comprising a reduced amount of the preservative, wherein reduced is relative to effective amount of the preservative within a similar composition devoid of PC (or comprising an active agent which is not PC).

In some embodiments, the effective amount of the preservative is determined based on a PET test, according to USP <51>, or any alternative PET test.

In some embodiments, the ophthalmic composition comprises between 0.001 and 1% w/w of a preservative, including any range between. In some embodiments, the ophthalmic composition comprises at least 0.001% w/w and below the label claim of the preservative, wherein below the label claim encompasses a concentration of the preservative being reduced by at least 10%, at least 50%, at least 70%, at least 2 times, at least 5 times, at least 10 times, at least 50 times, at least 100 times, at least 500 times, or between about 2 and about 20 times, between about 2 and about 15 times, between about 2 and about 10 times, between about 5 and about 20 times, between about 5 and about 15 times, including any range or value therebetween, relative to the label claim of the preservative.

In some embodiments, a w/w concentration of the preservative within the ophthalmic composition is between 0.001 and 1%, between 0.001 and 0.1%, between 0.001 and 0.01%, between 0.001 and 0.02%, between 0.001 and 0.018%, between 0.001 and 0.015%, between 0.002 and 0.018%, between 0.002 and 0.015%, between 0.003 and 0.018%, between 0.003 and 0.015%, between 0.05 and 0.1%, between 0.1 and 0.2%, between 0.2 and 0.3%, between 0.3 and 0.4%, between 0.4 and 0.5%, between 0.5 and 0.7%, between 0.7 and 1%, including any range or value therebetween. In some embodiments, the preservative is suitable for use in an ophthalmic composition. In some embodiments, the preservative is or comprises a quaternary ammonium cation, such as benzalkonium (BAK) including any salt thereof, for example benzalkonium chloride. In some embodiments, the quaternary ammonium cation is a pharmaceutically acceptable compound. In some embodiments, the quaternary ammonium cation comprises a pharmaceutically acceptable counter anion.

The inventors surprisingly found a synergistic preservative activity of the PC (specifically, phosphorylcholie-tuftsin conjugate of Formula 1, SEQ ID NO: 2) and BAK, resulting in significantly reduced concentration of BAK in the composition of the invention required for the sufficient preservative activity, relative to the label claim of BAK in ophthalmic preparations (0.02% w/w).

In some embodiments, the quaternary ammonium cation is represented by Formula NR₄+, wherein each R is independently selected from an alkyl group, an aryl group, an alkyl-aryl group, and wherein each of alkyl group, aryl group, and alkyl-aryl group is optionally substituted by one or more substituents each independently selected from —NO₂, —CN, —OH, oxo, imino, —CONH₂, —CONR′₂, —CNNR′₂, —CSNR′₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof.

As used herein, the term “alkyl” describes an aliphatic hydrocarbon including straight chain and branched chain groups. The alkyl group has between 1 to 50 carbon atoms, 1-30 carbon atoms, or 1-20 carbon atoms. Whenever a numerical range; e.g., “1-30”, is stated herein, it implies that the group, in this case the alkyl group, may contain 1, 2 carbon atoms, 3 carbon atoms, 4 carbon atoms, 5 carbon atoms, 6 carbon atoms, 7 carbon atoms, 10 carbon atoms, 15 carbon atoms, 20 carbon atoms, etc., up to and including 30 carbon atoms. In some embodiments, the alkyl group is a short alkyl comprising between 1 and 10, or between 1 and 6 carbon atoms, including any range between. In some embodiments, the alkyl group is a long alkyl having at least 10 carbon atoms, or between 10 and 30 carbon atoms including any range between. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein. The aryl group as used herein also encompasses a heteroaryl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms.

Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, 0-C₁₋₆-alkyl, C₁₋₆-alkyl, hydroxy-C₁₋₆-alkyl and amino-C₁₋₆-alkyl.

The term “alkylaryl” describes an alkyl, as defined herein, which is substituted by an aryl, as described herein. An exemplary alkylaryl is C₁₋₆-alkyl-aryl, e.g. benzyl.

In some embodiments, the quaternary ammonium is a polymer comprising one or more quaternary ammonium cations of Formula

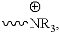

wherein each R is independently as disclose hereinabove, and wherein a wavy bond represents the attachment point to the polymeric backbone. Exemplary polymeric quaternary ammonium cations include but are not limited to Polyquaternium (e.g. Polyquaternium-2, Polyquaternium-80, Polyquaternium-11, Polyquaternium-52, Polyquaternium-17, etc.).

In some embodiments, the quaternary ammonium cation and/or salt thereof is selected from, without being limited thereto, benzyldimethyldodecylammonium chloride, didecyldimethylammonium chloride, dodecyltrimethylammonium chloride, hexadecyltrimethylammonium chloride, Sodium Cocamidopropyl PG-Dimonium Chloride Phosphate (Cola Lipid C), Laurdimoniumhydroxypropyl decylglucoside chloride (SugaQuat L-1010), Berberine, Berberrubine, Berberis vulgaris root extract, Isopropylbenzyl butylnorberberine iodide, Palmatine, Jatrorrhizine, Coptisine, Ungeremine, Epiberberine, Pseudoberberine, Stepharanine, Sinapine, and any combination thereof.

Additional preservatives are well-known in the art. Non-limiting examples of preservatives include but are not limited to, chlorobutanol, sodium perborate, and stabilized oxychloro complex (SOC) or any combination thereof.

In some embodiments, the preservative as disclosed herein is the only preservative in the ophthalmic composition of the invention. In some embodiments, the preservative within the ophthalmic composition is a benzalkonium, including any salt and any derivative thereof.

In some embodiments, the term “derivative” refers to a stereoisomer (e.g. enantiomer, and/or diastereomer) and/or a structural isomer (e.g. an alkylated, an animated, a hydroxylated, a carboxylated derivative), an ester, a tautomer, and/or any prodrug or precursor of the above disclosed compounds. In some embodiments, the term “derivative” refers to a structural derivative having a similar (or an enhanced) antimicrobial activity.

In some embodiments, the ophthalmic composition of the invention comprises below 0.02%, below 0.019%, or below 0.018% w/w of BAK or any derivative of BAK.

In some embodiments, the ophthalmic composition of the invention comprises between 0.001 and 0.018%, between 0.001 and 0.015%, between 0.001 and 0.01%, between 0.001 and 0.02%, between 0.001 and 0.018%, between 0.001 and 0.015%, between 0.002 and 0.018%, between 0.002 and 0.015%, between 0.003 and 0.018%, between 0.003 and 0.015% w/w, including any range or value therebetween.

In some embodiments, the ophthalmic composition comprises between 1 and 10%, between 1 and 3%, between 3 and 5%, between 5 and 7%, between 4 and 6%, between 7 and 10% w/w of a tonicity regulator including any range or value therebetween.

Non-limiting examples of tonicity regulators include, but are not limited to, mannitol, erythritol, inositol, xylitol, sodium chloride, potassium chloride, dextrose, glycerin, and propylene glycol or any combination thereof.

In some embodiments, the ophthalmic composition comprises about 4% w/w of mannitol.

In some embodiments, the ophthalmic composition comprises or consists essentially of: an aqueous solution (e.g. a buffered aqueous solution), between 0.1 and 5 w/w, between 0.1 and 2 w/w, between 0.1 and 3 w/w, between 0.1 and 1.5 w/w of phosphorylcholine-tuftsin conjugate, between 0.5 and 1.5% w/w of the mucoadhesive polymer (e.g. alginate), between 0.3 and 0.8% w/w of the viscosity enhancer (e.g. HPMC), about 4% w/w of the tonicity regulator (e.g. mannitol), and an effective amount of the preservative (e.g. BAK). Exemplary composition is as described hereinbelow. In some embodiments, the ophthalmic composition comprises or consists essentially of: an aqueous solution (e.g. a buffered aqueous solution), between 0.1 and 1.5 w/w of phosphorylcholine-tuftsin conjugate, between 0.5 and 1.5% w/w of the mucoadhesive polymer (e.g. alginic acid), between 0.3 and 0.8% w/w of the viscosity enhancer (e.g. HPMC), about 4% w/w of the tonicity regulator (e.g. mannitol), and between 0.001 and 0.019% w/w of the preservative (e.g. BAK). Exemplary composition is described hereinbelow.

In another aspect of the invention, there is provided an ophthalmic composition comprising or consisting essentially of: an aqueous solution (e.g. a buffered aqueous solution), between 0.5 and 1.5% w/w of the mucoadhesive polymer (e.g. alginic acid), between 0.3 and 0.8% w/w of the viscosity enhancer (e.g. HPMC), about 4% w/w of the tonicity regulator (e.g. mannitol), and an effective amount of the preservative of the invention, (i.e. a combination between a quaternary ammonium cation, such as BAK and the phosphorylcholine-tuftsin conjugate). In some embodiments, the effective amount of the preservative (also used herein as “the preservative effective concentration”) is reduced, compared to a preservative effective concentration of the quaternary ammonium cation within a similar composition devoid of PC, wherein “reduced” is as described hereinabove.

In some embodiments, the effective amount of the preservative within the ophthalmic composition of the invention corresponds to a concentration of the quaternary ammonium cation within between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between. In some embodiments, the effective amount of the preservative within the ophthalmic composition of the invention further comprises PC, wherein the w/w ratio of PC to the quaternary ammonium cation is as described herein. In some embodiments, the term “corresponds to”, when related to the concentration of the quaternary ammonium means that the concentration of the PC may vary, however the w/w ratio between the PC and the quaternary ammonium cation within the ophthalmic composition of the invention is as described hereinabove.

In some embodiments, the effective amount of the preservative within the ophthalmic composition of the invention further comprises a w/w ratio between the PC and the quaternary ammonium cation within said ophthalmic composition is between about 1000:1 and 1:1, between about 500:1 and 1:1, between about 700:1 and 1:1, between about 100:1 and 1:1, between about 50:1 and 1:1, between about 1000:1 and 10:1, between about 100:1 and 10:1, between about 500:1 and 10:1, between about 100:1 and 20:1, between about 50:1 and 10:1, between about 50:1 and 20:1, between about 10:1 and 5:1, between about 10:1 and 1:1, including any range between.

In some embodiments, the effective amount of the preservative within the ophthalmic composition of the invention comprises between 0.1 and 5 w/w, between 0.1 and 2 w/w, between 0.1 and 3 w/w, between 0.1 and 1.5 w/w of phosphorylcholine-tuftsin conjugate, including any range between.

In some embodiments, the ophthalmic composition consists essentially of pharmaceutical grade constituents. In some embodiments, the entire constituents of the ophthalmic composition are pharmaceutical grade compounds. In some embodiments, the ophthalmic composition is sterile. In some embodiments, the effective amount of the preservative within the ophthalmic composition of the invention is sufficient for maintain the sterility of the ophthalmic composition upon exposure thereof to non-sterile condition (e.g. ambient atmosphere comprising one or more microbes), wherein maintain is for a time period ranging between 1 and between 1 day and 2 months, between 1 day and lmonth, between 1 and 20 d, between 1 and 60 d, between 1 and 50 d, between 10 and 60 d, between 1 and 10 d, between 10 and 50 d, between 10 and 40 d, between 10 and 30 d, including any range between. The term “sterility” as used herein, refers inter alia to the microbial load of a composition (e.g. ophthalmic composition of the invention) or article according to the sterility requirements as recorded in US Pharmacopoeia, or in European Pharmacopoeia, respectively.

Methods for determining a level of appearance of a microorganism cells are known in the art.

In some embodiments, the pH of the ophthalmic composition disclosed herein is between 6 and 8, between 6 and 6.5, between 6.5 and 7, between 7 and 7.5, between 7.5 and 8, including any range or value therebetween.

In some embodiments, the ophthalmic composition is characterized by a viscosity between 30 and 150 cps, between 50 and 120 cps, between 1 and 5 cps, between 5 and 10 cps, between 10 and 15 cps, between 15 and 20 cps, between 20 and 30 cps, between 30 and 40 cps, between 40 and 50 cps, between 50 and 60 cps, between 60 and 70 cps, between 70 and 80 cps, between 80 and 90 cps, between 90 and 100 cps, between 100 and 110 cps, between 110 and 120 cps, including any range or value therebetween.

In some embodiments, the ophthalmic composition is characterized by a tonicity between 200 and 600 mOsmols/kg, between 200 and 500 mOsmols/kg, between 200 and 300 mOsmols/kg, between 300 and 350 mOsmols/kg, between 350 and 400 mOsmols/kg, between 400 and 450 mOsmols/kg, between 450 and 500 mOsmols/kg, between 500 and 550 mOsmols/kg, between 550 and 600 mOsmols/kg, including any range or value therebetween. In some embodiments, the ophthalmic composition is characterized by a tonicity between 250 and 450 mOsmols/kg.

According to other embodiments, the ophthalmic composition is a long acting, controlled release, extended release or slow release formulation (configured to prolong the release of PC, or to induce a release of PC at a predetermined release rate). Each possibility is a separate embodiment of the invention.

In some embodiments, the ophthalmic composition comprises an additive including, without limitation, a buffer component, a pH adjustor, and an electrolyte, and or any mixture thereof.

In some embodiments, the ophthalmic composition is in a form of an eye drop formulation.

In some embodiments, the ophthalmic composition is for use in the prevention or treatment of a disorder associated with ocular inflammation in a subject in need thereof.

In some embodiments, the ophthalmic composition is for use in enhancing ocular bioavailability of a phosphorylcholine-tuftsin conjugate in the subject.

### Preservative

In another aspect, there is provided a preservative comprising a phosphorylcholine-tuftsin conjugate (PC), including any salt thereof as described herein; and a quaternary ammonium cation including any salt thereof as described herein, wherein a w/w ratio (also referred to herein as “synergistically effective ratio”) between the PC and the quaternary ammonium cation within the preservative is between about 1000:1 and about 1:1 between about 500:1 and 1:1, between about 700:1 and 1:1, between about 100:1 and 1:1, between about 50:1 and 1:1, between about 1000:1 and 10:1, between about 100:1 and 10:1, between about 500:1 and 10:1, between about 100:1 and 20:1, between about 50:1 and 10:1, between about 50:1 and 20:1, between about 10:1 and 5:1, between about 10:1 and 1:1, including any range between. In some embodiments, the preservative is characterized by a synergistic antimicrobial activity when applied to a pharmaceutical or a cosmeceutical composition, corresponding to a concentration of the quaternary ammonium cation within the pharmaceutical or a cosmeceutical composition being below the label claim thereof. In some embodiments, the synergistic antimicrobial activity refers to the preservative or antimicrobial activity within an aqueous composition at a reduced effective preservative or antimicrobial concentration of the quaternary ammonium cation wherein reduced is as described herein.

In some embodiments, the quaternary ammonium cation is BAK. In some embodiments, the synergistic antimicrobial activity refers to the preservative or antimicrobial activity within an aqueous composition (e.g. pharmaceutical or a cosmeceutical composition) at an effective amount of BAK below 0.2 mg/ml, below 0.18 mg/ml, below 0.15 mg/ml, between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between.

In some embodiments, the preservative composition of the invention is in a form of a kit. In some embodiments, the constituents are at a synergistically effective ratio within the kit, wherein the synergistically effective amount is as described herein. In some embodiments, the kit comprises one or more quaternary ammonium cation(s) and one or more PC(s) including any salt thereof as the sole active ingredients, wherein at least one or all of the active ingredients are stored in separate container or packages.

In some embodiments, the kit further comprises an additive as disclosed hereinabove, wherein a w/w ratio between the additional agent and the active ingredients within the kit of the invention is between about 0.1:1 and 1:1, between about 0.2:1 and 1:1, between about 0.2:1 and 0.5:1, between about 0.1:1 and 0.5:1, including any range between, and optionally wherein (iii) is stored in a separate container.

In some embodiments, the kit is an antimicrobial, sanitizing, sterilizing or a preservative kit. In some embodiments, the kit comprises one or more compartments (e.g. a first compartment, a second compartment, and optionally a third compartment), wherein each of the compartments comprises one or more of the constituents of the kit, as disclosed herein. In some embodiments, the kit further comprises a carrier (e.g. an aqueous carrier) stored in a separate compartment.

In some embodiments, the kit comprises instructions for mixing of the first compartment, the second compartment, and optionally the third compartment, so as to obtain a composition comprising the constituents of the kit at a predefined ratio (e.g. a synergistically effective amount), as disclosed herein.

In some embodiments, the kit comprises instructions for dilution of the constituents of the kit (e.g. with an appropriate carrier, such as an aqueous solution) in an amount sufficient for obtaining a liquid composition comprising the effective concentration of the active ingredients.

In some embodiments, the kit comprises instructions for applying or contacting a predetermined amount of the kit to a predetermined amount of a liquid composition (e.g. pharmaceutical composition or cosmeceutical composition) to obtain the effective amount of the preservative within the liquid composition, wherein the effective amount is as described hereinabove. In some embodiments, the kit comprises instructions for applying or contacting a predetermined amount of the kit to a predetermined amount of a liquid composition to obtain the effective amount of the preservative within the liquid composition, corresponding to a reduced concentration of the quaternary ammonium cation within the liquid composition, wherein reduced is as described herein. In some embodiments, the quaternary ammonium cation is BAK (optionally wherein the PC is or comprises the compound of Formula 1, SEQ ID NO: 2), and the effective amount of the preservative corresponds to BAK concentration within the liquid composition below 0.2 mg/ml, below 0.18 mg/ml, below 0.15 mg/ml, above 0.01 mg/ml, including any range between.

In some embodiments, the kit comprises instructions for applying or contacting a predetermined amount of the kit to a predetermined amount of a liquid composition, so as to obtain BAK concentration within the liquid composition of between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between.

In some embodiments, the preservative is a composition (i.e. a preservative composition), wherein the PC and the quaternary ammonium cation are the sole preservative constituents within the composition. In some embodiments, the preservative composition is substantially devoid of an additional preservative, which is not a combination of PC and the quaternary ammonium cation.

In some embodiments, the present invention provides preservative composition having reduced toxicity, due to the reduced amount of the quaternary ammonium cation (e.g. BAK), wherein reduced is relative to the label claim of the quaternary ammonium cation.

In some embodiments, the preservative composition is a powderous composition. In some embodiments, the preservative composition is a solid composition. In some embodiments, the preservative composition is in a dry state. In some embodiments, the preservative composition is a solid antimicrobial composition. In some embodiments, the preservative composition is a solid at a temperature between −50° C. and below the decomposition point of any of the constituents (e.g. below 50 or 60° C.).

Herein throughout, by “composition” or “preservative composition”, which are use herein interchangeably, it is further meant to refer to a formulation. As used herein, the terms “formulation”, refer to a vehicle composition in the form of a solution, an emulsion, a lotion, a cream, a gel etc., that optionally further comprises physiologically acceptable carriers and/or excipients and optionally other chemical components such as cosmetically, cosmeceutically or pharmaceutically active agents (e.g., drugs). The formulation can optionally further comprise a carrier, and optionally additional active agents and/or additives.

). In some embodiments, the preservative composition is a semi-solid or a gel at a temperature between 5 and 95° C. In some embodiments, the c preservative composition is a flowable composition. In some embodiments, the preservative composition is a liquid or a flowable composition.

In some embodiments, the preservative composition of the invention comprises the active (or antimicrobial) ingredients as described hereinabove, and further comprises a carrier. In some embodiments, the carrier is a pharmaceutically acceptable carrier, cosmeceutically acceptable carrier, a food grade carrier, or any combination thereof. In some embodiments, the preservative composition of the invention is an aqueous composition. In some embodiments, the aqueous composition is in a form of a solution, emulsion, suspension or a dispersion.

In some embodiments, a w/w concentration of the acceptable carrier within the preservative composition is between 50 and 99%, between 60 and 99%, between 70 and 99%, between 80 and 99%, between 90 and 99%, between 50 and 70%, between 70 and 90%, including any range therebetween.

In some embodiments, the preservative composition of the invention is an aqueous solution comprising the active (or antimicrobial) ingredients substantially or fully dissolved therewithin. In some embodiments, the composition of the invention is a ready to use composition or is in a form of a dilutable concentrate. In some embodiments, the components in the formulation act in synergism.

In some embodiments, the term synergism, or any grammatical derivative thereof, is defined as the simultaneous action of two or more compounds in which the total biological activity (e.g. preservative effectiveness, and/or antimicrobial activity) of the combination is greater than the sum of the individual components.

In some embodiments, the preservative composition of the invention is characterized by sterilizing or preservation activity within a liquid composition (such as an aqueous pharmaceutical or cosmeceutical composition as disclosed hereinabove) or on a surface upon contacting a preservative effective amount of the preservative composition with the liquid composition or with the surface under suitable conditions (such as a temperature between 1° and 40° C., for a time period between 1 minute and 14 days, including any range between). In some embodiments, the preservation activity refers to a preservative effectiveness, as determined by Preservative Effectiveness Testing (PET) according to USP<51>.

In some embodiments, the liquid composition and/or the surface refers to liquid composition or surface prone of microbial infestation (such as articles comprising a water content of at least 10%, at least 50%, or between 10 and 99%, between 20 and 90%, between 20 and 80%, between 30 and 99%, including any range between).

In some embodiments, the preservative composition or the kit of the invention is formulated for use in a liquid composition or on a surface susceptible to microbial growth as a preservative. In some embodiments, the liquid composition is selected from a cosmeceutical article, a pharmaceutical article, and/or a food-grade article.

In another aspect of the invention, there is provided a pharmaceutical composition comprising an effective concentration of the preservative of the invention, wherein effective concentration is as described herein. In some embodiments, the pharmaceutical composition is an ophthalmic composition. In some embodiments, the pharmaceutical composition is prone to microbial infestation. In some embodiments, the pharmaceutical composition is a liquid, a flowable composition, a semi-solid, as semi-liquid, a foam, or a gel. The terms “effective concentration” and “effective amount” are used herein interchangeably, and refer to the preservative or antimicrobial effective concentration of the preservative of the invention.

In some embodiments, the effective concentration is a preservative effective amount. In some embodiments, the preservative effective amount corresponds to a reduced concentration of the quaternary ammonium cation within the pharmaceutical composition, wherein reduced is as described hereinabove. In some embodiments, the effective concentration corresponds to a concentration of the quaternary ammonium cation below 0.18 mg/ml within the pharmaceutical composition. In some embodiments, the effective concentration corresponds to a concentration of the quaternary ammonium cation (e.g. BAK) between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between.

In some embodiments, the effective concentration comprises a concentration of the quaternary ammonium cation (e.g. BAK) between 0.01 and 0.19 mg/ml, between 0.01 and 0.18 mg/ml, between 0.01 and 0.15 mg/ml, between 0.01 and 0.1 mg/ml, between 0.02 and 0.18 mg/ml, between 0.02 and 0.15 mg/ml, between 0.015 and 0.18 mg/ml, between 0.015 and 0.15 mg/ml, between 0.03 and 0.18 mg/ml, between 0.03 and 0.15 mg/ml, including any range between, and further comprises the PC, wherein a ratio between PC and quaternary ammonium cation (e.g. BAK) is as described hereinabove (i.e. between about 1000:1 and about 1:1, including any range between).

In some embodiments, the effective concentration comprises a concentration of the quaternary ammonium cation as described hereinabove, and a concentration of the PC between 1 and 50 mg/ml, including any range between.

The term “antimicrobial” refers to a composition or compound capable of preventing the growth, inhibiting the growth, and/or controlling the growth of microorganisms; antimicrobial compounds include bactericides, bacteriostats, fungicides, fungistats, algaecides and algistats. The term “antimicrobial” encompasses preservatives and sterilization agents.

In some embodiments, the term “antimicrobial activity” is referred to as an ability to inhibit (prevent), reduce or retard bacterial growth, fungal growth, biofilm formation or eradicate living bacterial cells, or their spores, or fungal cells or viruses in a suspension or in a moist environment.

Herein, inhibiting or reducing or retarding the formation of load of a microorganism refers to inhibiting, reducing, or retarding growth of microorganisms and/or eradicating a portion or all of an existing population of microorganisms. Thus, formulations described herein can be used both in reducing the formation of microorganisms on or in an article, and in killing microorganisms in or on an article or a living tissue.

The microorganism can be, for example, a unicellular microorganism (prokaryotes, archaea, bacteria, eukaryotes, protists, fungi, algae, molds, yeast, euglena, protozoan, dinoflagellates, apicomplexa, trypanosomes, amoebae and the likes), or a multicellular microorganism.

In some embodiments, the microorganism comprises bacterial cells of bacteria such as, for example, Gram-positive and Gram-negative bacteria.

In some embodiments, the Gram-positive bacteria are Staphylococcus aureus, Staphylococcus epidermidis, and Bacillus cereus.

In some embodiments, the Gram-negative bacteria are Escherichia coli, Pseudomonas aeruginosa, and Burkholderia cepacia.

In some embodiments of the present invention, the fungi is Candida albicans.

In some embodiments, the mold is Aspergillus brasiliensis.

The term “biofilm”, as used herein, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaea, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and fungi. The cells of a microorganism growing in a biofilm may be physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

The term “antibiofilm” refers to the capacity of a substance to disturb the formation of a biofilm of bacterial, fungal and/or other cells, and/or to affect a reduction in the rate of buildup of a biofilm of bacterial, fungal and/or other cells, on a surface of a substrate.

As used herein, the term “preventing” in the context of antimicrobial or preservative, indicates that the growth rate of the microorganism cells is essentially nullified or is reduced by at least 20%, at least 30%, at least 40%, at least 50%, at least 60%, at least 70%, at least 80%, at least 90%, including any value therebetween, of the appearance of the microorganism in a comparable situation lacking the presence of the preservative of the invention or an article containing same. Each possibility represents a separate embodiment of the invention. Alternatively, preventing means a reduction to at least 15%, 10%, or 5% of the appearance of the microorganism cells in a comparable situation (e.g. an aqueous composition) lacking the presence of the preservative.

As used herein, the term “reducing” in the context of preservation activity or effectiveness, indicates that the growth rate (and or microbial loading expressed in CFU or CFU/ml) of the microorganism (including spores, cells, or biofilm thereof) is essentially reduced as compared to a similar article devoid of the preservative of the invention. In some embodiments, the term “essentially reduced” comprises at least 2 times, at least 5 times, at least 10 times, at least 50 times, at least 100 times, at least 1000 times, at least 10.000 times, at least 100.000 times, at least 1000.000 times, between 10 and 1000.000 times, between 100 and 1000.000 times, between 1000 and 10.000.000 times, between 1000 and 1000.000 times, between 10.000 and 1000.000 times CFU/ml reduction, including any range between, as compared to the CFU/ml content of a similar composition devoid of the preservative of the invention (also referred to herein as the initial microbial loading). In some embodiments, the preservative substantially prevents microbial infestation of the article. In some embodiments, the article comprising the preservative composition of the invention maintains its sterility upon exposing thereof to non-sterile conditions (e.g. ambient atmosphere comprising one or more microbes) for a time period between 1 day and 2 months, between 1 day and lmonth, between 1 and 20 d, between 1 and 60 d, between 1 and 50 d, between 10 and 60 d, between 1 and 10 d, between 10 and 50 d, between 10 and 40 d, between 10 and 30 d, including any range between.

### Method

In another aspect, there is a method for treating or preventing an ocular disease or disorder in a subject in need thereof, the method comprising administering to an eye of the subject an effective amount of the ophthalmic composition of the invention. In some embodiments, the ocular disease comprises an ocular inflammation. Is an auotoimmune disease.

In another aspect, there is provided a method for enhancing ocular bioavailability of a phosphorylcholine-tuftsin conjugate (PC) in a subject afflicted with the ocular disease or disorder, comprising administering to an eye of the subject an effective amount of the ophthalmic composition of the invention, thereby enhancing a concentration of said PC in said eye. In some embodiments, effective amount of the ophthalmic composition refers to a therapeutically effective amount of the PC. The a therapeutically effective amount (or daily dosage) is further described hereinbelow.

In some embodiments, administering comprises administering to the subject a reduced dose of the PC, wherein reduced is as described hereinbelow. In some embodiments, a reduced dose of the PC is sufficient for obtaining or inducing a therapeutically effective concentration of the PC in the eye of the subject. In some embodiments, therapeutically effective concentration of the PC is sufficient for reducing at least one symptom associated with the ocular disease or disorder.

In some embodiments, the subject is afflicted with an inflammation. In some embodiments, the inflammation is an ocular inflammation. In some embodiments, the subject is afflicted with a disease or disorder selected from dry eye, dry macular degeneration, diabetic macular edema, and post operation inflammation. In some embodiments, ocular inflammation is uveitis.

In some embodiments, the method is for treating or preventing a disease or a disorder associated with ocular inflammation.

As used herein, the term “ocular inflammation” refers to any inflammation of any part of the eye. In some embodiments, the inflammation is of the middle layer of the eye. In some embodiments, the inflammation is uveitis. In some embodiments, the ocular inflammation comprises dry eye or dry macular degeneration. In some embodiments, the ocular inflammation is associated with another disease.

Non-limiting examples of systemic diseases (and/or autoimmune disease) which can result in ocular inflammation are Crohn's disease, Bechet disease, and Juvenile idiopathic arthritis. In some embodiments, the ocular inflammation is associated with an adverse reaction to a drug or environmental trigger. Non-limiting examples of such drugs include Rifabutin, quinolones, vaccines and allergens. In some embodiments, the ocular inflammation is associated with post operation inflammation. Non-limiting examples of such include post-cataract surgery, laser eye surgery and corneal transplantation.

The term “uveitis” as used herein refers to inflammation of the uvea, the pigmented inner layer that lies between the retina and the sclera and cornea. The inflammation can occur in the iris, ciliary body, choroid or any part of the uvea. Uveitis can refer to any one of anterior uveitis, intermediate uveitis, posterior uveitis, and pan uveitis.

As used herein, the terms “treatment” or “treating” of ocular inflammation encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, treating ocular inflammation comprises at least one of preventing the onset of ocular inflammation, attenuating the progress of ocular inflammation and inhibiting the progression of ocular inflammation.

In some embodiments, treating comprises reducing inflammation. In some embodiments, treating comprises reducing abnormal inflammation. In some embodiments, treating comprises reducing inflammation in an eye of the subject.

In some embodiments, treating comprises reducing secretion of at least one pro-inflammatory cytokine. In some embodiments, treating comprises stimulating the mammal's immunomodulation activity. A variety of models known to those skilled in the art may be used to establish the treatment efficacy of the ophthalmic composition of the invention. Cell cultures may be used to test the effect of the phosphorylcholine tuftsin-conjugates on cell proliferation, cytokine profile, development of tolerogenic dendritic cells and development of T regulatory cell. For example, commercial kits for following pro- and anti-inflammatory cytokine, including, but not limited to IL-1, IL-2, IFNγ, IL-4, IL-10, IL-15, IL-17 and TNFα may be used. Animal models, as are known to a person skilled in the art and as exemplified herein below, may be used to test the activity of the ophthalmic composition of the invention in treating, preventing or reducing the progression of autoimmune diseases.

In some embodiments, the method comprises a preliminary step preceding the administering step; wherein the preliminary step comprises determining a subject suitable for treatment by the PC and/or by the ophthalmic composition of the invention. In some embodiments, determining comprises determining a subject afflicted with an inflammatory disease (e.g. ocular inflammation, and/or an autoimmune disease disclosed herein, such as uveitis); (ii) selecting a subject afflicted with the inflammatory disease. In some embodiments, determining is by analyzing the presence and/or a concentration of an inflammation biomarker (e.g. a pro-inflammatory cytokine, including, but not limited to IL-1, IL-2, IFNγ, IL-4, IL-10, IL-15, IL-17 and TNFα) in a sample derived form the subject. In some embodiments, an increased concentration (at least 10% above the concentration in a healthy subject) of the inflammation biomarker is indicative for the subject being suitable for treatment by the composition of the invention.

According to some embodiments, the method of the invention comprises the steps of (i) determining or identifying a subject afflicted with an inflammatory disease; (ii) selecting a subject afflicted with the inflammatory disease; and (iii) treating the subject afflicted with the inflammatory disease with the ophthalmic composition of the invention.

According to some embodiments, the method of the invention comprises the steps of (i) identifying a subject in a risk for an autoimmune disease or afflicted with an autoimmune disease; and (iii) treating the subject with the ophthalmic composition of the invention.

In some embodiments, reducing comprises at least a 10%, 20%, 30%, 40%, 50%, 60%, 70%, 80%, 90%, 95%, 99% or 100% reduction. Each possibility represents a separate embodiment of the invention. It will be understood by one skilled in the art that each cytokine need not be reduced by the same amount. Some cytokines may be reduced by more than others.

In another aspect of the invention, there is a method for enhancing ocular bioavailability of a phosphorylcholine-tuftsin conjugate in a subject, comprising administering to an eye of the subject the ophthalmic composition of the invention.

In some embodiments, enhancing ocular bioavailability is by at least 10% compared to a control. In some embodiments, enhancing is by at least 10%, at least 20%, at least 30%, at least 40%, at least 50%, at least 60%, at least 70%, at least 80%, at least 90%, at least 95%, at least 99%, at least 100%, at least 200%, at least 500%, at least 1000%, at least 5000%, at least 10.000%, at least 100.000%, including any range or value therebetween.

In some embodiments, the method is for prolonging residence time of phosphorylcholine-tuftsin conjugate on or within an eye (e.g. cornea and/or aqueous humor). In some embodiments, prolonging is for a time period of 10 min, 20 min, 30 min, 40 min, 50 min, 60 min, 80 min, 100 min, 2 h, 3 h, 4 h, 5 h, including any value there between.

In some embodiments, the control or the control solution is an aqueous solution of phosphorylcholine-tuftsin conjugate, having about the same concentration of the phosphorylcholine-tuftsin conjugate as the composition of the invention. In some embodiments, the control solution is devoid of the mucoadhesive polymer and of the viscosity enhancer.

In some embodiments, enhancing ocular bioavailability comprises increasing concentration of the phosphorylcholine-tuftsin conjugate in an aqueous humor of the eye. In some embodiments, increasing is by at least 10%, at least 20%, at least 30%, at least 40%, at least 50%, at least 60%, at least 70%, at least 80%, at least 90%, at least 95%, at least 99%, at least 100%, at least 200%, at least 500%, at least 1000%, at least 5000%, at least 10.000%, at least 100.000%, including any range or value therebetween. As exemplified hereinbelow (Example 1), phosphorylcholine-tuftsin conjugate administered within the composition of the invention showed enhanced in-vivo accumulation within an aqueous humor.

In some embodiments, the subject is selected from a human subject and an animal subject.

As used herein, the terms “administering”, “administration”, and like terms refer to any method which, in sound medical practice, delivers a composition containing an active agent to a subject in such a manner as to provide a therapeutic effect. In some embodiments, the administering is ocular or intraocular.

The administering step may be repeated as needed to provide effective lubrication to such eye. The mode of administration of the present composition depends on the form of the composition. For example, if the composition is a solution, drops of the composition may be applied to the eye, e.g., from a conventional eye dropper. In general, the present compositions may be applied to the surface of the eye in substantially the same way as conventional ophthalmic compositions are applied.

A person skilled in the art will appreciate that eye drops in particular, will not perfectly reach the site of inflammation. As such the dose will need to be increased or decreased as determined by a skilled artisan to compensate for the mode of administration.

In some embodiments, the amount (dose) of a composition to be administered will, of course, be dependent on the subject being treated, in the medical condition being treated for, the severity of the affliction, the manner of administration, the judgment of the prescribing physician, etc.

In some embodiments, the daily dose (i.e. the amount of the phosphorylcholine-tuftsin conjugate per day) is between 50 and 2000 μg, between 200 and 2000 μg, between 50 and 100 μg, between 100 and 200 μg, between 200 and 300 μg, between 300 and 400 μg, between 400 and 500 μg, between 500 and 600 μg, between 600 and 700 μg, between 700 and 800 μg, between 800 and 900 μg, between 900 and 1000 μg, between 1000 and 1100 μg, between 1100 and 1300 μg, between 1300 and 1500 μg, between 1500 and 1800 μg, between 1800 and 2000 μg, including any range or value therebetween.

In some embodiments, the composition is administered 1, 2, 3, 4, 5, 6, 8, 9, 10 times a day, including any range therebetween.

The pharmaceutical composition may further comprise additional pharmaceutically active or inactive agents such as, but not limited to, an anti-bacterial agent, an antioxidant, a buffering agent, a bulking agent, a surfactant, an anti-inflammatory agent, an anti-viral agent, a chemotherapeutic agent and anti-histamine.

According to an embodiment of the present invention, the pharmaceutical composition described herein above is packaged in a packaging material and identified in print, in or on the packaging material, for use in the treatment of a disease or disorder, as described herein.

According to another embodiment of the present invention, the pharmaceutical composition is packaged in a packaging material and identified in print, in or on the packaging material, for use in monitoring a disease or disorder, as described herein.

Products of the present invention may, if desired, be presented in a pack or dispenser device, such as an U.S. Food and Drug Administration (FDA) approved kit, which may contain one or more unit dosage forms containing the disclosed composition. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the FDA for prescription drugs or of an approved product insert.

In some embodiments, the kit contains a single dosage form, wherein the dosage form contains a daily dose of the disclosed composition. In some embodiments, the kit contains a plurality of dosage forms. In some embodiments, the kit contains a plurality of dosage forms, wherein the plurality of dosage forms are equivalent to a daily dose of the disclosed composition.

In some embodiments, the pharmaceutical composition is characterized as being adhesive to a mucosal tissue (e.g. cornea). In some embodiments, the pharmaceutical composition is characterized by an increased residence time on or within the eye. In some embodiments, the pharmaceutical composition increases residence time of the phosphorylcholine-tuftsin conjugate within the eye or on top of the eye (e.g. on the cornea). In some embodiments, the pharmaceutical composition enhances corneal penetration of the phosphorylcholine-tuftsin conjugate.

In some embodiments, the pharmaceutical composition enhances chemical stability of the phosphorylcholine-tuftsin conjugate under physiological conditions (e.g. upon interaction with cornea and/or within aqueous humor).

The term “mucoadhesive”, or any grammatical derivative thereof, as used herein refers to the phenomenon where a substance applied to a mucosal epithelium (e.g. cornea), adheres, usually creating a new interface, to the mucus layer.

In some embodiments, the method is for extending the release period on or within the eye of the phosphorylcholine-tuftsin conjugate. In some embodiments, phosphorylcholine-tuftsin conjugate is slowly released. In some embodiments, phosphorylcholine-tuftsin conjugate is released in a controlled manner.

In this context, the term “controlled manner” indicates that the drug is released substantially constantly. Herein, the term “constantly” may refer to a time duration of about e.g., 10 min, 20 min, 30 min, 40 min, 50 min, 60 min, 80 min, 100 min, 2 h, 3 h, 4 h, 5 h, including any value there between.

It is understood that the composition of the present invention may be administered in conjunction with other drugs, including other anti-inflammatory drugs.

### General

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide”describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R″′ end group or a —R′NC(N) NR″— linking group, as these phrases are defined hereinabove, where R′, R″ and R″′ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove.

In some embodiments, the substituents are halo, hydroxy, cyano, O—C1-6-alkyl, C1-6-alkyl, hydroxy-C1-6-alkyl and amino-C1-6-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

As used herein the term “about” refers to ±10% or ±20%. Further, the entire numerical values disclosed herein are approximations also encompassing variations of ±10% or ±20% from the disclosed values. It is to be understood, that all numerical values disclosed herein are preceded by the term “about”.

The terms “comprises”, “comprising”, “includes”, “including”, “having” and their conjugates mean “including but not limited to”.

The term “consisting of” means “including and limited to”.

The term “consisting essentially of” means that the composition, method or article (e.g. fiber of the invention, or an article processed therefrom) may include additional ingredients, steps and/or parts, but only if the additional ingredients, steps and/or parts do not materially alter the basic and novel characteristics of the claimed composition, method or article. Further, the term “consisting essentially of” is used to define articles or compositions which include the recited elements but exclude other elements that may have an essential significance on the article or on the composition.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a” “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

### Materials

Materials used in the manufacture of the tested ophthalmic solutions are as follows: TRS (Drug Substance, Investigational grade); Monobasic sodium phosphate, dihydrate (Buffering Agent, USP/NF); Dibasic sodium phosphate dihydrate (Buffering Agent, USP/NF); Benzalkonium Chloride (Preservative, USP/NF); Sodium Alginate (mucoadhesive, Ph Eur); Hypromellose (Viscosity Enhancer, USP/NF); Sodium Hydroxide and/or Hydrochloric Acid (pH Adjusting agent, USP/NF); Water for Injection (Solvent, USP/NF); Mannitol (Tonicity regulator, USP).

Commercially available Lipicare® ready to use formulation was used as received.

Drug Substance [TRS]: is a trifluoroacetate [TFA] salt of phosphorylcholine-tuftsin conjugate represented by Formula 1, SEQ ID NO: 2 (as shown herein). Molecular formula: C43H68N13013P, Appearance: Yellow to orange solid. Solubility: >40 mg/ml in water.

Packaging of the ophthalmic compositions: single use media bottles (PETG) with HDPE screw caps. After filling the closure is sealed with tamper-proof labels. Bottles and closures are purchased radiation-sterilized and non-pyrogenic. Storage conditions: −20° C.±5° C. Long term stability study of [TRS] at −20° C.±5° C. and an accelerated stability study at ±5° C.±3° C., started recently for 36 months. Available stability data at −20° C.±5° C. proved that the drug substance is stable for 6 months if stored under these conditions.

### Methods

The target dosage of 0, 30, 150, or 300 g is delivered in a 30 L topical ocular drop of 0%, 0.1%, 0.5%, and 1% w/v TRS ophthalmic solution, respectively correlates the TRS formulations with the clinical dosage strengths in a 30 L drop (mean drop volume measured from 10 drops was 30.6 L).

During the development process, three lead formulation prototypes (See Table 1) were evaluated. Each lead formulation included some, or all, of the following: preservative agents, osmotic adjusters, viscosity enhancers, and excipients to improve the bioavailability of the drug. Each formulation was tested using a single ocular topical administration in albino rabbits, to evaluate the irritant properties, if any, and to provide ocular bioavailability of TRS.

In vivo experiments were performed as follows:

Nine male New Zealand White rabbits were randomly divided into three groups of 3 animals per time point: 30, 60, and 120 minutes after a 50 μl drop administration.

Aqueous humor (AH) from both eyes of each animal of each group at each time point, were sampled, weighed, snap-frozen and stored at −80°±15° C.

Samples preparation: The aqueous humors were extracted with an ice cold Acetonitrile/Methanol (50:50) solution containing the internal standard (propranolol, 100 nM) in a 1 to 5 ratio (V/V). After centrifugation (12 000 rpm, 10′, 4° C.), the supernatants were transferred in Matrix tubes for LC-MS/MS analysis.

Aqueous humor samples were analyzed using LC-MS (LC-MS/MS Acquity I-Class—Xevo TQS, Waters) as follows:

Column: Waters® Acquity BEH C18, 50*2.1 mm, 1.7 μm (40° C.). Mobile Phase: A: Ammonium Formate 5 mM, pH 3.75; B: CH3CN+Ammonium Formate 5 mM, pH 3.75, 5% aqueous. Injection volume: 0.2 μL. Flow: 600 μL/min.

### Gradient:

| Time (min) | B(%) |
|---|---|
| 0.00 | 2% |
| 0.20 | 2% |
| 2.00 | 98% |
| 2.50 | 98% |
| 2.60 | 2% |
| 4.00 | 2% |

### Source Temperature: 150° C. Desolvation Temperature: 600° C. Cone Gas Flow: 50 L/h.

Desolvation Gas Flow: 1200 L/h.

### Mass Spectrometry Characterization:

| | | | Capillary | | Cone | | Collision |
|---|---|---|---|---|---|---|---|
| | Molecular | | voltage | Parent | Voltage | Product | Energy |
| ID_Structure | Weight | Mode | (kV) | Ion | (V) | Ion | (eV) |
| TRS | 1006.07 | ES+ | 0.5 | 336.36 | 30 | 129.01 | 18 |
| (quantification | | | | | | | |
| trace) | | | | | | | |
| TRS | 1006.07 | ES+ | 0.5 | 336.36 | 30 | 69.96 | 32 |
| (identification | | | | | | | |
| trace) | | | | | | | |
| Propranolol | 259.34 | ES+ | — | 260.14 | 26 | 116 | 16 |
| (IS = Internal | | | | | | | |
| Standard) | | | | | | | |

TABLE 1
Exemplary formulations used in the rabbit in-vivo PK studies

| Formulation # | Composition |
|---|---|
| 1 | TRS (5% w/v), Phosphate buffer (10 mM), |
| | HPMC (0.5%), BAK (0.02%), Mannitol |
| | (1.7%) |
| 2 | TRS (0.94% w/v), Lipicare ® (Micro-emulsion |
| | technology for delivering ophthalmic drugs) |
| 3 | TRS (1.0%, w/v), Phosphate buffer (10 mM), |
| | HPMC (0.5%), Sodium Alginate (1.4%), BAK |
| | (0.02%), Mannitol (3.9%) |

Exemplary Formulation 3 was characterized as follows:

Ophthalmic Solution is a sterile, viscous solution of a water-soluble synthetic molecule [TRS]. The formulation is buffered with sodium phosphate salts targeting a range around physiological pH [6.5-7.5] and is rendered isotonic with mannitol [250-400 mOsm]. Benzalkonium chloride is added as a preservative. Sodium alginate and hypromellose (HPMC) were added to increase the viscosity of the solution and to prolong ocular retention time and enhance penetration of the TRS01 into the eye. WFI is the solvent.

### Example 1

Formulation 3 was tested for homogeneity and was found homogenous. GLP ocular tolerability studies for 28 days were conducted in NZW rabbits and beagle dogs using Formulation 3; and was found to be both safe and well-tolerated in both animal models.

All formulations were well-tolerated after single topical ocular administration in albino rabbits and exhibited a Cmax of TRS at 30 minutes following instillation. Formulation 2 exhibited the poorest ocular penetration of TRS. There was no bioavailability advantage for Formulation 1, which had the highest TRS concentration. Formulation 3, having both ionic mucoadhesive polymer (sodium alginate) and the viscosity enhancer, significantly enhanced TRS bioavailability in-vivo, compared to Formulation 1 and Formulation 2.

### Example 2

The inventors evaluated the synergistic preservative activity of the combination of TRS (Formula 1) and benzalkonium chloride. In brief, 1.0% w/w TRS formulations based on the constituents of Formulation 3, with various concentration of benzalkonium chloride have been prepared. Subsequently, the solutions have been subjected to the Preservative Effectiveness Testing (PET) according to USP<51>.

TRS formulation that contained only 0.003% w/w benzalkonium chloride (corresponding to 15% of the label claim) passed PET test. Furthermore, TRS formulation with even lower concentration of benzalkonium chloride of 0.001% (corresponding to 5% label claim) showed a solid synergistic antimicrobial effect. A similar formulation without benzalkonium chloride didn't show any significant antimicrobial effect, pointing out the synergistic effect of both benzalkonium chloride and TRS in the preservative composition of the invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-8. (canceled)

9. An ophthalmic composition comprising an aqueous solution, comprising (i) a pharmaceutically effective amount of a phosphorylcholine-tuftsin conjugate (PC), (ii) between 0.1 and 5% weight per weight (w/w) of a viscosity enhancer, and (iii) between 0.1 and 5% w/w of a mucoadhesive polymer.

10. The ophthalmic composition of claim 9, wherein said ophthalmic composition further comprises a quaternary ammonium cation at a preservative effective concentration below a preservative effective concentration of the quaternary ammonium cation within a similar composition devoid of said PC.

11. The ophthalmic composition of claim 10, wherein said quaternary ammonium cation is or comprises benzalkonium (BAK), including any salt thereof.

12. The ophthalmic composition of claim 10, wherein said preservative effective concentration of said quaternary ammonium cation within said ophthalmic composition is between 0.01 and 0.18 mg/ml.

13. The ophthalmic composition of claim 10, wherein a w/w ratio between the PC and the quaternary ammonium cation within said ophthalmic composition is between about 1000:1 and 1:1.

14. The ophthalmic composition of claim 9, wherein a w/w ratio between said mucoadhesive polymer and said viscosity enhancer is between 1:1 and 10:1.

15. The ophthalmic composition of claim 9, wherein said pharmaceutically effective amount is between 0.01 and 10% w/w of said phosphorylcholine-tuftsin conjugate.

16. The ophthalmic composition of claim 9, wherein said phosphorylcholine-tuftsin conjugate comprises at least one phosphorylcholine moiety or a derivative thereof and tuftsin or a derivative thereof covalently linked via a spacer; wherein said spacer comprises at least two amino acids; and wherein said phosphorylcholine moiety or a derivative thereof is covalently linked to said spacer via a diazo group.

17. (canceled)

18. (canceled)

19. The ophthalmic composition of claim 9, wherein said phosphorylcholine-tuftsin conjugate is represented by Formula 1:

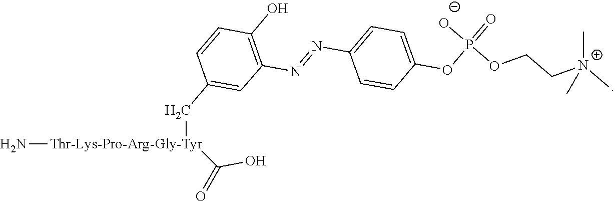

20. The ophthalmic composition of claim 9, wherein said mucoadhesive polymer comprises an ionic mucoadhesive polymer.

21. The ophthalmic composition of claim 20 wherein said ionic mucoadhesive polymer is selected form the group consisting of: alginic acid, chitosan, polyacrylate, hyaluronic acid, carboxymethylcellulose, pectin, and gelatin including any salt, any copolymer, or any combination thereof.

22. The ophthalmic composition of claim 9, wherein said viscosity enhancer comprises hydroxy methyl cellulose, hydroxy ethyl cellulose, cellulose, hydroxypropyl methyl cellulose, polyvinylpyrrolidone, polyethyleneglycol, polypropyleneglycol, poly(2-hydroxyethyl methacrylate), and polyvinylalcohol, including any salt, any copolymer, or any combination thereof.

23. (canceled)

24. The ophthalmic composition of claim 9, further comprises up to 5% w/w of a tonicity regulator; and wherein the ophthalmic composition is in a form of an eye drop formulation formulated for ocular administration.

25. (canceled)

26. A method for treating or preventing an ocular disease or disorder in a subject in need thereof, the method comprising administering to an eye of said subject an effective amount of the ophthalmic composition of claim 9.

27. The method of claim 26, wherein the ocular disease or disorder comprises an ocular inflammation.

28. The method of claim 27, wherein said ocular inflammation is uveitis, or a condition associated with any one of dry eye, dry macular degeneration, and post operation inflammation.

29. A method for enhancing ocular bioavailability of a phosphorylcholine-tuftsin conjugate (PC) in a subject afflicted with an ocular disease or disorder, comprising administering to an eye of said subject the ophthalmic composition of claim 9, thereby enhancing a concentration of said PC in said eye.

30. The method of claim 29, wherein said enhancing ocular bioavailability is by at least 10% compared to a control; and wherein said enhancing ocular bioavailability comprises increasing concentration of said phosphorylcholine-tuftsin conjugate in an aqueous humor of said eye.

31. (canceled)

32. The method of claim 26, wherein said administering comprises administering to said subject a reduced dose of said PC, thereby obtaining a therapeutically effective concentration of said PC in said eye, wherein said therapeutically effective concentration is sufficient for reducing at least one symptom associated with said ocular disease or disorder; and wherein reduced dose comprises at least 10% reduced amount of said PC compared to a control.

33. The method of claim 32, wherein said method comprises a preliminary step preceding said administering; wherein the preliminary step comprises determining a subject suitable for treatment by said PC; and wherein said subject is selected from a human subject and an animal subject.


SEQUENCE LISTING (2 sequences)

SEQ ID NO: 1 - protein, 4 aa, synthetic construct
TKPR

SEQ ID NO: 2 - protein, 6 aa, synthetic construct
TKPRGY